(19) United States
(12) Patent Application Publication
(10) Pub. No.: US 2011/0130756 A1
(43) Pub. Date: Jun. 2, 2011

(54) VASCULATURE DEVICE

(76) Inventors: David C. Everson, JR., Chesapeake City, MD (US); David C. Keach, Newark, DE (US); Benjamin M. Trapp, Flagstaff, AZ (US); Neil R. Williams, Lincoln University, PA (US)

(21) Appl. No.: 12/945,652

(22) Filed: Nov. 12, 2010

Related U.S. Application Data
(60) Provisional application No. 61/265,501, filed on Dec. 1, 2009.

(51) Int. Cl. A61B 18/00

(52) U.S. Cl. 606/41

(57) ABSTRACT

A vasculature device with a wire with a shaped set portion, an electrically conductive path, an electrical connector connecting the wire to the electrically conductive path at a point distal to the shaped set portion, and a hypotube encasing the wire, electrically conductive path and electrical connector is provided. The vasculature device can be actuated from a low-profile configuration to a deployed configuration by heating of the wire, and in particular the shaped set portion of the wire, via application of an electrical current to the wire. The vasculature device is useful in removal of clots, thromboemboli and foreign bodies from the vasculature, and in particular the cerebral vasculature, and as steering wires or guidewires.

### CROSS REFERENCE TO RELATED APPLICATIONS

This application claims priority to U.S. Provisional Application No. 61/265,501 filed on Dec. 1, 2009, the entirety of which is incorporated herein by reference.

### FIELD OF THE INVENTION

The present invention relates to vasculature devices useful, for example, in removing objects such as thrombus or other foreign bodies from a patient's vasculature. More particularly, the invention relates to devices useful for removing thrombus from a patient's cerebral vasculature. Devices of the present invention can also be used as steerable guidewires or steering wires in the vasculature of a patient.

### BACKGROUND OF THE INVENTION

The use of a mechanical means to restore patency to obstructed vessels is well known. These devices fit into many categories ranging from hydraulic removal of thrombus, rotating cutting blades for calcified plaque, inflatable means for crushing or dragging thrombus, or a multiplicity of metal structures that either self-expand or can be expanded to dredge a vessel or remove a stone.

Examples of these devices date back to the ‘Fogarty Catheter’ described by Fogarty in U.S. Pat. Nos. 3,367,101; 3,435,826; and 4,403,612 describing in detail improvements to a balloon catheter for embolectomy purposes. While suitable for many applications, dragging a balloon through the delicate, tortuous cerebral vasculature could cause unwanted trauma, Crossing profiles of current state of the art balloon catheters would also limit their use with typical neurovascular accessories (e.g., microcatheters).

Mechanically expanded devices are also well known in the area of obstruction removal. Clark specifically focused on the use of an expanding braid for thrombus removal in U.S. Pat. No. 3,996,938. His teaching utilized a braid that would expand under the force of compression delivered by an inner core wire affixed to the distal end of the braid.

Many refinements on this theme have occurred in the areas of stone removal, clot removal, foreign body removal, etc. All of these are assemblies of some nature which either self-expand or mechanically expand under some delivered compressive load. Examples of these can be seen in Bates U.S. Pat. No. 6,800,080 in which parallel legs of the basket allow bodies to enter the retrieval basket; Bates U.S. Pat. No. 5,496,330 in which the basket is self-expanding and meant to collapse into a provided sheath; Engelson U.S. Pat. No. 6,066,158 describing a self-expanding conical basket held collapsed in a ‘delivered’ state because of a ‘fixedly attached core wire’; and Samson U.S. Pat. No. 6,066,149 describing an assembly consisting of a series of braided expanders.

These devices, while elegant, fail to address the major concern for applications into the neurovasculature, namely, minimizing the crossing profile (i.e., the cross-sectional area) of the devices. In general, these are all assembled devices consisting of many components that need to either be welded in place, or fixedly attached using collars, etc. It is not seen how a device of these inventions would be compatible with physician preferred microcatheters used to access the delicate, tortuous neurovasculature.

In many of the inventions, the issue of crossing profile has been circumvented by describing fixed wire assemblies which are not meant to pass through a microcatheter, rather, they are meant to navigate from a large guiding catheter situated well proximal of the obstruction in large vasculature. Samson U.S. Pat. No. 6,066,149 is an example of this type of assembly. As demonstrated in the figures, the device is an assembly in which the wire ends are managed into a collar. The retractable core wire doubles as a conventional guidewire tip at its distal termination. This tip affords the steering of a guidewire and the ability to puncture a clot to cross it, while the large body of the device encompasses the expander. Perhaps suitable for easily accessible obstructions, this does not address the majority of anticipated cerebral vascular cases, or the physician preference, where a microcatheter/guidewire combination is used to create a pathway across the clot for angiographic visualization distal to the clot prior to the procedure.

Wensel has anticipated the need for smaller devices to achieve neurovasculature compatibility in U.S. Pat. No. 5,895,398. In this publication, he teaches the use of a helically shaped wire held straight for delivery by the microcatheter. By using a single wire shaped into a ‘cork-screw’ he has circumvented the complex assembly steps required in much of the other prior art resulting in large profiles. His invention, unfortunately, places the need of restraint on the microcatheter. Typically, physician preferred microcatheters are extremely flexible at the distal end lending little ability to hold a shaped wire straight. This results in a trade-off of making the ‘cork-screw’ floppy (which degrades its ability to extract a clot), or making a custom microcatheter which is stiff, limiting procedural access.

In U.S. Pat. No. 5,895,398 and in the subsequent publication U.S. Pat. No. 6,436,112, Wensel describes a coil type device for removing clots and foreign bodies in vessels wherein the coil is made out of a biphasic material which changes shape upon heating or the passage of an electric current. The coil is described as straight initially, and then after passing an electrical current or heat, the coil changes to its coil configuration. The coil is attached to an insertion mandrel. No means for passage of an electrical current or heat are described.

### SUMMARY OF THE INVENTION

The invention relates to vasculature devices and methods of making and using the same.

The vasculature device comprises a wire with a shaped set portion, an electrically conductive path extending parallel to the wire, at least one electrical connector between the wire and the electrically conductive path distal to the shaped set portion of the wire, and a hypotube into which the wire, conductive path, and electrical connector are inserted.

The wire in the vasculature device of the present invention comprises a biphasic material which changes shape in the shaped set portion upon heating of the wire via an electrical current.

In one embodiment, the vasculature device is a mechanical thrombectomy device. In this embodiment, the shaped set portion of the wire is straight initially. Upon heating the shaped set portion forms a coil useful in capturing clots, capturing and removing thromboemboli and removing other foreign bodies in the vasculature, and in particular the cerebral vasculature.

In another embodiment, the vasculature device is used as a steerable guidewire or steering wires in the vasculature. In this embodiment, any desired shape for use as a guidewire or steerable wire may be envisioned.

### DESCRIPTION OF THE DRAWINGS

The operation of the present invention should become apparent from the following description when considered in conjunction with the accompanying drawings, in which:

FIG. 1 provides a schematic illustration of an wire of a vasculature device of the present invention with the shaped set portion in a straight, low-profile configuration.

FIGS. 2a through 2c provide a schematic illustration of one embodiment of an electrically conductive path running parallel to the wire of FIG. 1. FIG. 2a illustrates a first insulating layer of an electrically conductive path surrounding the wire of the device. FIG. 2b illustrates the first insulating layer surrounding the wire depicted in FIG. 2a and an electrically conductive member extending along the outer surface of the first insulating layer of the device. FIG. 2c illustrates the first insulating layer surrounding the wire, the electrically conductive member extending along the outer surface of the first insulating layer, and a second insulating layer covering the electrically conductive member of the device.

FIG. 3a provides a schematic illustration of an embodiment of the hypotube component of the device.

FIG. 3b provides a schematic illustration of an embodiment of the device of the present invention enclosed in a hypotube. It should be understood that the figure illustrates one surface of a tubular structure.

FIG. 3c provides a schematic illustration of an alternative embodiment of the device of the present invention enclosed in a hypotube. It should be understood that the figure illustrates one surface of a tubular structure.

FIG. 4 provides a schematic illustration of an occluded artery.

FIG. 5 provides a schematic illustration of an occluded artery with a mechanical thrombectomy device of the present invention inserted through the occlusion.

FIG. 6 provides a schematic illustration of the deployment of the shaped set portion of the wire of the thrombectomy device of the present invention into a coiled configuration for capture of the clot within the occluded artery.

FIG. 7 provides a schematic illustration of the clot of FIG. 4-6 being removed from the artery via the thrombectomy device of the present invention.

### DETAILED DESCRIPTION OF THE INVENTION

Provided by the present invention are vasculature devices.

Vasculature devices of the present invention comprise a longitudinally extending wire with a shaped set portion, an electrically conductive path extending parallel to the wire, an electrical connector connecting the wire and the electrically conductive path at a point distal to the shaped set portion of the wire, and a hypotube into which the wire, conductive path and electrical connector are inserted.

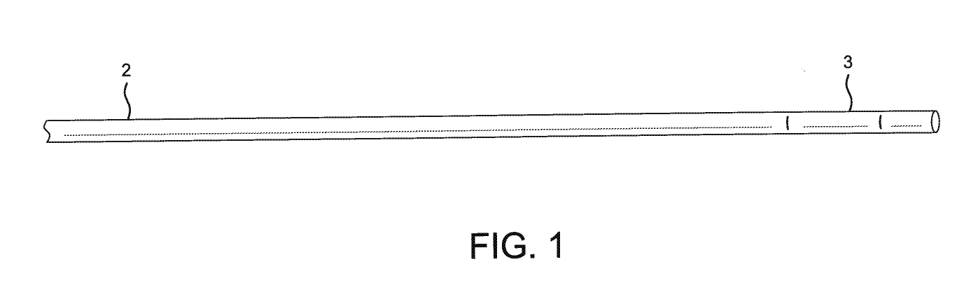

Elements of the vasculature device of present invention are depicted in FIGS. 1 through 3.

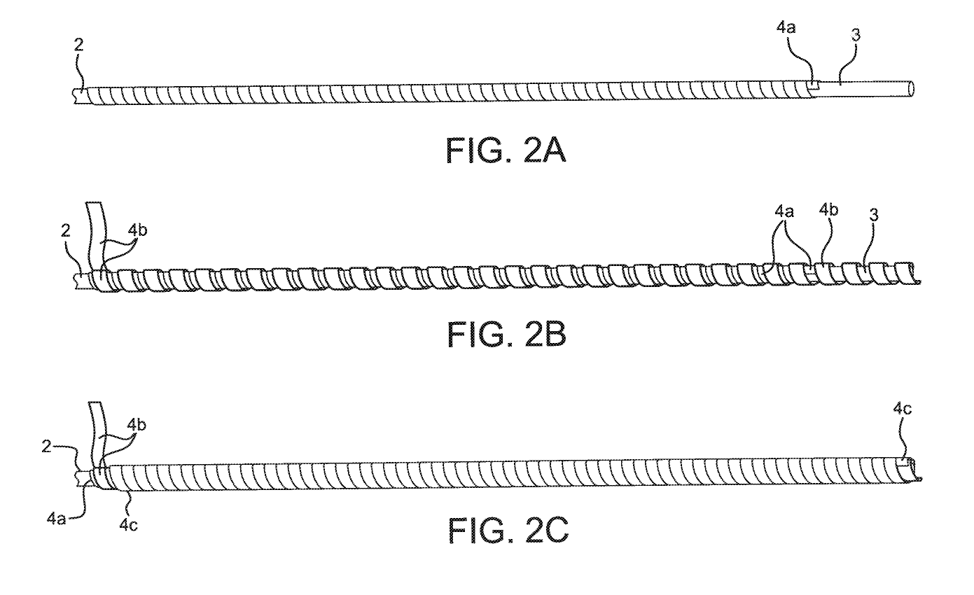

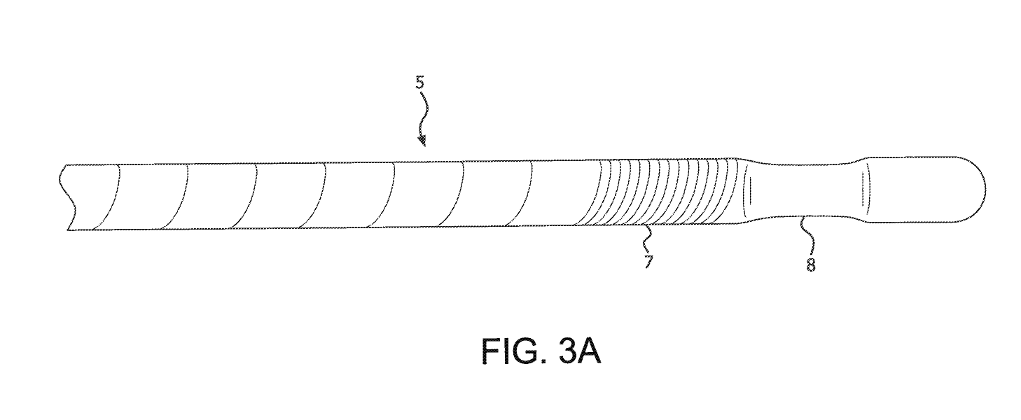

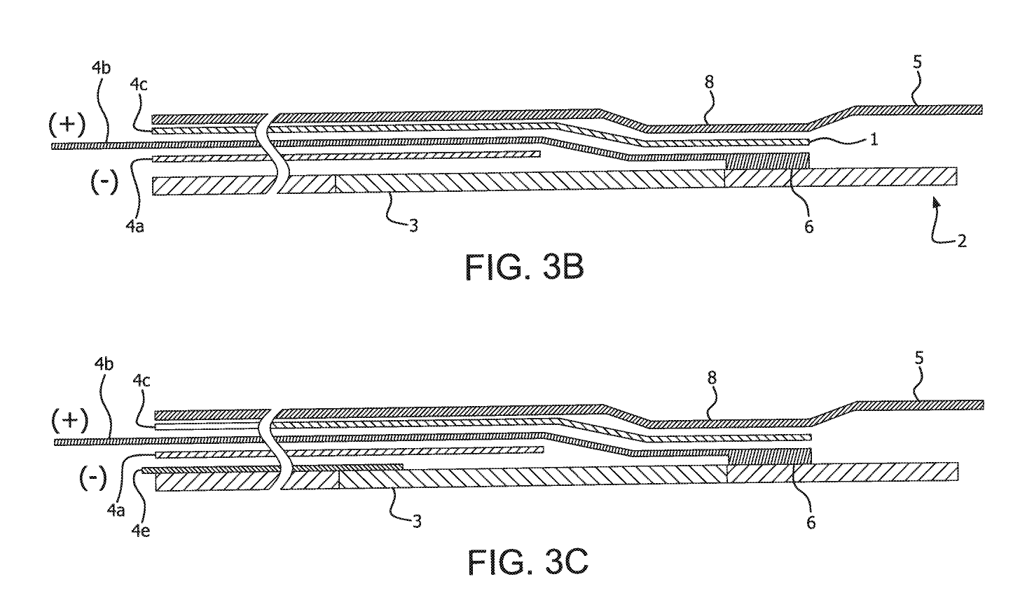

Specifically, FIG. 1 is illustrative of the longitudinally extending wire 2 with a shaped set portion 3 of the device. The wire 2 is comprised of a biphasic material which changes shape in the shaped set portion 3 upon heating of the wire. In FIG. 1 as well as FIGS. 2 and 3, the shaped set portion 3 of the wire 2 is depicted in a straight or low profile configuration.

An example of biphasic material useful as the wire 2 of the device of the present invention is Nitinol, which has its metallurgy tailored to transform to Austenite at a temperature around or above body temperature, but below what causes damage to the surrounding tissue. Experiments in heat treating a nitinol wire in accordance with the present invention resulted in an Aₛ of approximately 33° C. and an A_{f} of approximately 42° C. While the Nitinol wire begins transformation to an austenitic state (Aₛ) at a temperature below body temperature, the gradient does not build enough force to overpower the hypotube and change shape until or above A_{f}, where Nitinol finishes its transformation to an austenitic state. Any biphasic material such as a shape set polymer, would be of use in this application.

Diameter of the wire may be varied and is dependent upon the electrically conductive path, internal diameter of the hypotube, and the needed strength of the coil. The larger the diameter of the wire, the stronger the coil will be. In one embodiment, the diameter of the wire is the largest diameter which can be wrapped in an electrically conductive path and still fit into the hypotube. The largest diameter wire which fits is selected to provide maximum strength to the shaped set portion of the wire. For example, the inventors herein found that for a hypotube with an internal diameter (ID) of 0.0093″ or 236 mm, a 0.005″ or 127 mm to 0.006″ or 152 mm diameter Nitinol wire that could include an electrically conductive path and fit into the hypotube would be preferable. The preferred length of wire would be about 180 cm or mimic the length of the preferred guidewire.

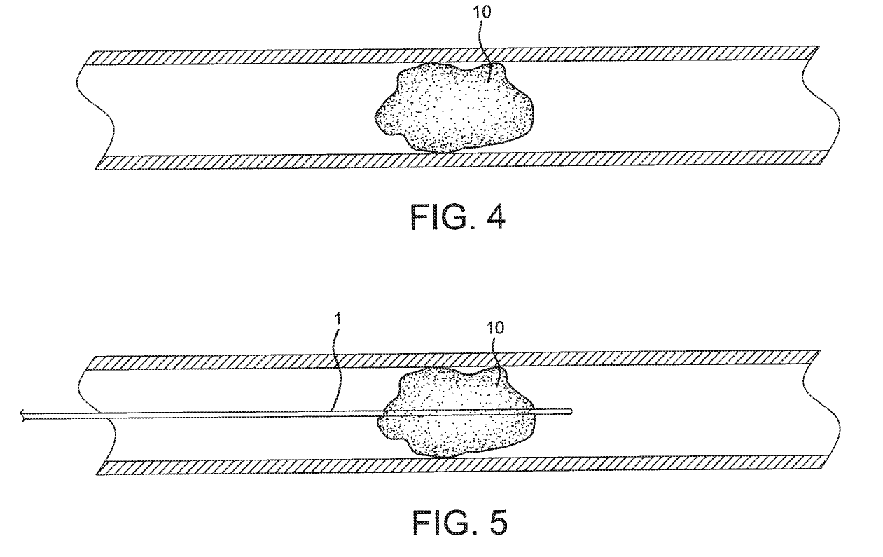

In FIGS. 4-7, depicting an embodiment of a vasculature device 1 of the present invention useful as a mechanical thrombectomy device, the shaped set portion 3 of the wire 2 is depicted as a coil configured to capture clots, filter, capture and remove thromboemboli and capture and remove foreign bodies from the vasculature. Various coil configurations including, but in not way limited to straight coils and tapered coils can be used.

In one embodiment, the shaped set portion 3 of the wire 2 is built into the wire by heat conditioning the wire as it is wrapped around a mandrel of a size selected for use of the vasculature device.

For example, for a mechanical thrombectomy device, a 3.2 mm mandrel with a 1.6 mm mandrel support (a fixture which holds the ends of the mandrel) provides for 2 full revolutions of the wire at a known pitch resulting in a straight coil. Use of a mandrel such as this eliminates kinking where the wire enters and exits the mold as the 1.6 mm mandrel support acts as a gentle lead in.

Alternatively, a tapered coil can be built into the wire of a mechanical thrombectomy device. A tapered coil may provide additional resistance to coil straightening under clot load as well as added recovery force to overpower a stiffer hypotube.

The wire 2 in the vasculature device of the present invention is heated via Joule heating as a result of an electrical current being passed through the wire. A voltage is supplied across the system via an attached direct current source such as a battery. A resulting current flows from the “+” (positive) terminal of the voltage source through the wire 2 (−) as shown in FIG. 3b. The current is returned to the “−” (negative) terminal of the voltage source via a low resistance path 4e which is electrically connected 6 to the wire 2 at a point distal to the shape set portion 3 as illustrated in FIG. 3C.

FIGS. 2a through 2c are illustrative of one embodiment of an electrically conductive path 4 extending parallel to the wire 2. In this embodiment, the electrically conductive path comprises a first insulating layer 4a surrounding the wire of the device (see FIG. 2a). The electrically conductive path 4 of this embodiment further comprises an electrically conductive member 4b extending along the outer surface of the first insulating layer 4a (see FIG. 2b). The first insulating layer 4a isolates the electrically conductive member from the wire except at the desired electrical connection distal to the shaped set portion of the wire. The electrically conductive path 4 of this embodiment may further comprise a second insulating layer 4c covering the electrically conductive member 4b. This second layer would electrically isolate the hypotube from the conductive path and provide a barrier to contaminants. In this embodiment, the first and/or second insulating layers may comprise more than one layer to ensure complete isolation of the electrically conductive member from the wire except at the desired electrical connection distal to the shaped set portion of the wire and/or to prevent contaminates (such as blood and water) from disrupting the insulation.

Various materials can be used for the layers of this embodiment of an electrically conductive path.

For example, for the electrically conductive member, a thin metal containing film or foil capable of conducting an electrical current with a resistance lower than the biphasic material of the wire, such as aluminized/polyester (PET) film or foil can be used. Any other thin, malleable conductive material could be used including a conductive metallic braid. By using a film or foil with a resistance lower than the wire, the majority of the power delivered goes towards heating the wire. Thickness of the film is dependent upon wire diameter and the internal diameter of the hypotube into which the wire and electrically conductive path must be fitted and the desired resistance. In one embodiment, a film layered with the aluminum at 0.00035″ or 0.0089 mm and PET at 0.00048″ or 0.012 mm thickness resulting in a total thickness of 0.00083″ or 0.0096 mm is used.

For the first insulating layer, a thin film wrap capable of isolating the wire from the electrically conductive member except for at the desired electrical connection is selected. For example, a thin film wrap of an expanded polytetrafluoroethylene (ePTFE composite) such as ePTFE/ethylene fluorinated ethylene propylene (EFEP) (approx. 0.0001″ or 0.0025 mm thick) can be used. This composite has a low melt thermoplastic layer which reflows during heating to fix the film in place and further prevent liquids from entering the system. Any thermoplastic composite such as FEP (fluorinated ethylene propylene), PE (polyethylene), or Pebax® combined with any low melt thermoplastic may be used in this application. The insulating layer is applied between the wire and the electrically conductive member to prevent the wire from contacting the electrically conductive member except where desired. A second insulating layer of the same or different material can be applied on top of the electrically conductive member to prevent contact between the hypotube and the electrically conductive member and to prevent contaminates (such as water or blood) from disrupting the insulation.

At a point distal to the shaped set portion 3 of the wire 2, the wire 2 and the electrically conductive path 4, and in particular the electrically conducting member 4b of the electrically conductive path, must come in contact to allow the circuit to be completed. This point of contact is referred to herein as an electrical connector 6. In the electrically conductive path embodiment of FIGS. 4a through 4c, electrical connection is achieved by stopping the first insulating layer 4a at a point distal to the shaped set portion of the wire but proximal to the distal end of the electrically conductive member 4b and compressing the wire and conductive member together via a crimp in the hypotube. Alternative methods to a crimp of creating an electrical connection such as a silver epoxy could be used.

An alternate conductive path may be envisioned. This alternate configuration is shown in FIG. 3c. It is similar in construction to the configuration shown in FIGS. 2a to 2c except that it contains an additional conductive film layer 4e. The conductive film layer 4e has a lower resistance than that of the wire 2 which causes only the shape set portion to heat and therefore assume its preset shape.

The vasculature device of the present invention further comprises a hypotube 5 which encases the entire wire and electrically conducting path (see FIG. 3b). See FIGS. 3a through 3c. The hypotube provides strength for insertion of the vasculature device at the proximal end while maintaining flexibility at the distal end to form the shape of the shaped set portion of the wire after heating. Any conductive, thin, instrument of adequate mechanical properties could be used in place of a hypotube. In one embodiment, the hypotube comprises a stainless steel tube with an internal diameter (ID) selected to be adequate to encase the wire and electrically conductive path and an outer diameter (OD) selected to fit within the microcatheter and vasculature, in particular the cerebral vasculature. In one embodiment, a fine pitch spiral is machined into at least a portion of the hypotube at its distal end adjacent to the shaped set portion of the wire to allow for distal flexibility upon shape change of the wire. See FIGS. 3a and 3b. In one embodiment, the hypotube comprises a section of uncut tube distal to the shaped set portion of the wire wherein a crimp is placed to electrically connect via compression the electrically conducting path to the wire at a point distal to the shaped set portion of the wire.

Vasculature devices of the present invention are compatible with physician-preferred accessories (e.g., microcatheters) allowing for easier, more rapid access to vascular anatomy, and in particular the cerebrovascular anatomy such as, but not limited to, M1 and M2 levels of the Mid-Cerebral Artery (MCA), distal Internal Carotid Artery (ICA), and Vertebral-Basilar Arteries, as compared to devices requiring use of a specific microcatheter or cumbersome microcatheter exchange. Further, actuation of the vasculature device of the present invention is straightforward and quick as compared to devices requiring a complex system of rotations and/or counter rotations to, for example, engage a clot sufficiently. Fewer procedural manipulations provide for shorter procedural time and are advantageous to the patient.

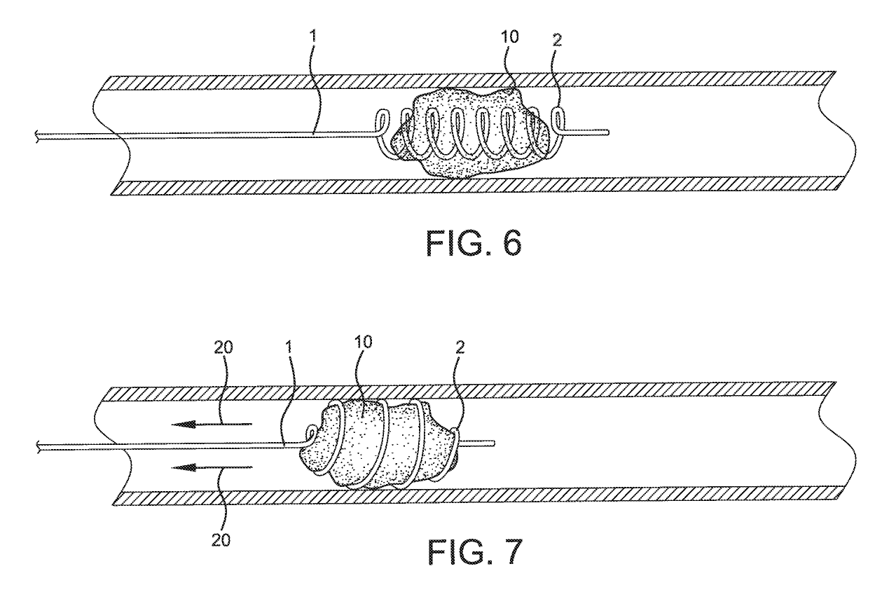

One embodiment of the vasculature device of the present invention, as depicted in FIG. 4 through 7, provides an efficient mechanical thrombectomy device for the vasculature, and in particular the cerebral vasculature. The thrombectomy device of the present invention is useful in removal of clots, capture and removal of thromboemboli and removal of foreign bodies from the vasculature. This thrombectomy device is illustrated within an artery in FIGS. 4 through 7. For use of this embodiment, a patient presenting symptoms of a thromboembolic disorder is examined to confirm diagnosis and locate the occlusion. A large introducing catheter is then inserted into an appropriate vessel such as the femoral artery or femoral vein. A small physician-preferred microcatheter is then introduced into the vessel via the introducing catheter and advanced using, for example, a guidewire into the occluded vessel. The device is then advanced through the clot to a sight distal of the clot. The mechanical thrombectomy device of the present invention is then advanced through the physician-preferred microcatheter in a low-profile configuration to the site of the clot. The mechanical thrombectomy device of the present invention is then further advanced in a low-profile configuration through the viscoelastic clot to a point where the shaped set portion of the wire of the mechanical thrombectomy device is at the clot site (see FIG. 5). The mechanical thrombectomy device of the present invention is then actuated via passage of an electrical current through wire of the device to assume a deployed configuration of the shaped set portion of the wire and encasing hypotube (see FIG. 6). In the deployed configuration, the thrombectomy device has a sufficient geometry and mechanical attributes to engage and remove clots with a minimum of embolized debris (see FIG. 7). Efficient clot capture and removal provide for shorter procedural durations, and improved re-vascularization in the patient. In the deployed configuration, the thrombectomy device can also be used to capture and remove thromboemboli and to remove other foreign bodies and could be used as a steering wire or guidewire.

### EXAMPLES

Without intending to limit the scope of the invention, the following examples illustrate how various embodiments of the invention may be made and/or used.

A vasculature device similar to FIGS. 3a,3b, and 3c was manufactured using the following components and assembly process.

The following components were used.

A nitinol wire (Fort Wayne Metals, Fort Wayne, Ind.) with a diameter of 0.005″ (0.127 mm) which had a section of the distal end heat set to a straight coil (no taper) with an Aₛ of about 33° C. and an A_{f} of about 42° C. The wire was shape set using shape set heat treatment techniques commonly known in the art.

A stainless steel laser cut spiral hypotube (Creganna, Marlborough, Mass.) with an ID and OD of, respectively, 0.0093″×0.0132″ (0.236×0.335 mm). The pitch of the spiral cut was specified. A 2 mm portion near the distal end of the hypotube was left uncut to provide a location suitable for crimping.

A 0.005″ (0.127 mm) stainless steel process mandrel.

A length of aluminum/polyester foil slit to 0.015″ (0.381 mm) width provided from in house stock.

A length of EFEP (Ethylene Fluorinated Ethylene Propylene) film slit to 0.030″ (0.762 mm) width provided from in house stock.

Loctite® 460 (Henkel Corp., Rocky Hill, Conn. 06067) adhesive.

Shrink tube (part #008025CST, 0.008″ (0.203 mm) ID, Advanced Polymers, Inc. Salem, N.H. 03079).

Sand paper, 400 grit.

A platinum coil with the following dimensions, 0.009″×0.006″×12″ (0.228×0.152×304.8 mm)

A tape wrapping machine was used in the manufacturing of the following device. Specific speed, angle and tension settings used are further described where needed.

Sand paper was used to strip an about 2 cm section of oxide from the wire just distal of the shaped set portion. The wire was bent in half at a place approximately 170 cm distal from the proximal section of coil shaped region. The bent portion of the wire and the straight portion of the wire were secured in the tape wrapping machine.

The tape wrapping machine was set with the following specifications for the first insulating layer of EFEP tape wrapping. Mandrel speed was set to 2000 rpm in the reverse rotation direction. Wrap angle was set to 61°, the mandrel tension was set to 3 psi the payoff tension to 0 psi, the traverse direction was set to right to left. EFEP tape was loaded onto the wrapping machine and wrapped by hand around the wire four times leaving a tag length at the proximal end of the wire. A soldering iron was used to secure the hand wrapped tape to the wire. The tag end of the tape was then trimmed by hand. The wrapping machine was engaged and the wire wrapped until just before the region of oxide removal on the wire. The soldering iron was again used to secure the tape to the wire and the excess tape was trimmed close to the mandrel. The wire was removed from the tape wrapping machine and baked in a 165° oven for three minutes. The wire was then removed from the oven and placed back in the tape wrapping machine.

The tape wrapping machine was set to the following specifications for the electrical conducting member. Mandrel speed was set to 800 rpm in the reverse direction. Wrap angle was set to 57.8°, the mandrel tension was set to 3 psi, the payoff tension to 5 psi and the traverse direction to right to left. Aluminum/polyester foil was loaded onto the tape wrapping machine and laid over the wire about 1 cm distal of where the first EFEP layer started. The end of the foil was wrapped four times around the wire and taped to secure with masking tape. The wrapping machine was engaged and the wire wrapped with foil until about 1 cm past the distal end of the first EPEP layer making sure contact between the core wire and the foil is made at the oxide stripped section of core wire. A drop of Loctite® adhesive was placed under the foil directly on the wire to secure foil to the wire. The excess foil was trimmed by hand as close to the wire as possible.

A second insulating layer of EFEP was applied with the following settings to the tape wrapping machine. Mandrel speed was set to 2000 rpm in the reverse direction. Wrap angle was set to 53°, the mandrel tension was set to 3 psi, the payoff tension to 0 psi and the traverse direction to right to left. EFEP tape was loaded onto the tape wrapping machine and laid over the wire about 5 mm distal of the proximal end of the foil layer. The tape was wrapped around the wire four times and secured and trimmed in the same manner as the first EFEP layer. The tape wrapper was engaged and the wire wrapped with tape until about 5 cm past the end of the foil layer. The tape was then secured and trimmed in the same manner as the first tape layer, removed from the wrapping machine and baked in an oven set to 165° for three minutes.

After removal from the oven, the distal unwrapped end of the wire was straightened by hand and inserted into the proximal (uncut) end of the hypotube until the distal end of the wire assembly protruded past the distal end of the hypotube. The proximal end of the wire assembly was clamped onto a working surface. The distal end of the wire was pulled by hand to straighten the wire. The hypotube was positioned by hand proximally over the wire until the foil was exposed at the distal end of the hypotube.

The distal end of the wire was trimmed by hand at about 5 mm from the distal end of the foil. A 1 cm section of shrink tube was positioned over the cut end of the core wire to a position that would cover the foil section with the shrink tube. The shrink tube was then heated to melt the distal end of the shrink tube. Excess shrink tube was trimmed by hand to a point about 1 mm past the wire. The end of the shrink tube was reheated.

A 5 mm section of platinum coil was cut and positioned over the distal end of the wire assembly leaving a 2-3 mm space between foil and platinum coil. A small drop of glue was placed onto the wire and the coil was moved on the core wire up to the foil portion. The platinum coil provides improved radiopacity to the device.

Excess glue was removed by hand. The proximal end of the hypotube and the wire were held by hand and the wire was pulled for a couple of millimeters. The hypotube was relaxed by hand to remove any compression and the platinum coil was positioned inside the hypotube but pushing gently by hand. The wire was pulled until the distal end was inside the hypotube by about 1 mm. Care was taken to ensure that about 1 mm of the nonspiral cut section of hypotube was about 2-3 mm distal of the shaped region of the wire.

The distal end of the assembly was inserted into a crimping collete, leaving about 0.5 mm of uncut hypotube outside of the collete. The collete was tightened by hand. A 15 mm wrench was used to tighten the collete ¼ of a turn. 1 mm of the distal tip of the hypotube was crimped by hand and the 15 mm wrench was used to tighten the collete ⅛ of a turn. The assembly was removed from the collete.

What is claimed is:

1. A vasculature device comprising:
(a) a longitudinally extending wire having a proximal end, a distal end and a shaped set portion;
(b) at least one electrically conductive path extending parallel to said wire;
(c) an electrical connector connecting said wire to said electrically conductive path at a point distal to said shaped set portion of said wire; and
(d) a hypotube encasing said wire, said electrically conductive path and said electrical connector.

2. The vasculature device of claim 1 wherein the wire comprises a biphasic material which changes shape in the shaped set portion upon heating of the wire via an electrical current

3. The vasculature device of claim 1 wherein the wire comprises nitinol.

4. The vasculature device of claim 1 wherein the electrically conductive path comprises:
(i) a first insulating layer at least partially surrounding the wire and extending to a point distal to the shape set portion; and
(ii) an electrically conductive member extending along at least a portion of an outer surface of the first insulating layer to a point distal to the first insulating layer and in contact with the wire distal to the shape set portion.

5. The vasculature device of claim 4 wherein the first insulating layer comprises an expanded polytetrafluoroethylene (ePTFE) composite.

6. The vasculature device of claim 5 wherein the ePTFE composite comprises silk and ethylene fluorinated ethylene propylene.

7. The vasculature device of claim 4 wherein the electrically conductive member comprises a thin metal containing film or foil capable of conducting an electrical current with a resistance lower than the wire.

8. The vasculature device of claim 7 wherein the electrically conductive member comprises an aluminized/polyester (PET) film or foil.

9. The vasculature device of claim 4 further comprising a second insulating layer covering at least a portion of the electrically conductive member and extending to a point distal to the electrically conductive member and in contact with the wire.

10. The vasculature device of claim 9 wherein the first insulating layer comprises an expanded polytetrafluoroethylene (ePTFE) composite.

11. The vasculature device of claim 10 wherein the ePTFE composite comprises silk and ethylene fluorinated ethylene propylene.

12. The vasculature device of claim 1 wherein a portion of the hypotube is cut for flexibility.

13. The vasculature device of claim 12 wherein the hypotube comprises a section of uncut tube distal to the shaped set portion of the wire.

14. The vasculature device of claim 13 wherein a crimp is placed in the uncut section to electrically connect via compression the electrically conducting path to the wire at a point distal to the shaped set portion of the wire.

15. The vasculature device of claim 1 wherein the shaped set portion of the wire forms a coil upon heating of the wire.

16. The vasculature device of claim 15 wherein the coil has a sufficient geometry and mechanical attributes to engage and remove clots from the vasculature.

17. The vasculature device of claim 15 wherein the coil has a sufficient geometry and mechanical attributes to filter, capture and remove thromboemboli from the vasculature.

18. The vasculature device of claim 15 wherein the coil has a sufficient geometry and mechanical attributes to remove foreign bodies other than clots and thromboemboli in the vasculature.

19. The vasculature device of claim 1 further comprising a means for conducting an electrical current through the wire.

20. A mechanical thrombectomy device comprising the vasculature device of claim 1 wherein the shaped set portion of the wire forms a coil upon heating of the wire with geometry and mechanical attributes to engage and remove clots from the vasculature.

21. A method for removing a clot from the vasculature comprising:
inserting into an appropriate vessel a large introducing catheter;
introducing into the vessel via the introducing catheter a small physician-preferred microcatheter;
advancing the microcatheter to the occluded vessel;
advancing, via the physician-preferred microcatheter, the thrombectomy device of claim 20 to the site of the clot, said thrombectomy device being in a low-profile configuration;
further advancing said thrombectomy device in the low-profile configuration through the clot to a point where the shaped set portion of the wire of the mechanical thrombectomy device is at the clot site;
passing an electrical current through the wire of the thrombectomy device so that the thrombectomy device assumes a deployed configuration; and
capturing the clot in the thrombectomy device so that the clot is removed from the vasculature upon removal of the thrombectomy device.

22. A steering wire or guidewire comprising the vasculature device of claim 1.
